(19) United States
(12) Patent Application Publication
(10) Pub. No.: US 2007/0248557 A1
(43) Pub. Date: Oct. 25, 2007

(54) Mild leave-on composition

(75) Inventors: Brent William Mason, West Chester, OH (US); Robert Ya-lin Pan, Cincinnati, OH (US); Shellie Jean Porter Caudill, Liberty Township, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The IAMS Company

(21) Appl. No.: 11/788,416

(22) Filed: Apr. 20, 2007

Related U.S. Application Data
(60) Provisional application No. 60/794,894, filed on Apr. 25, 2006.

(51) Int. Cl. A61K 8/73; A61K 8/36; A61K 8/46; A01N 25/00

(52) U.S. Cl. 424/70.13; 424/70.22; 424/70.24; 424/70.21; 424/405

(57) ABSTRACT

The present invention relates to a leave on companion animal composition having a composition having a Total Lather Volume of from about 300 ml to about 700 ml, IL1α of from about 0 pg/ml to about 7000 pg/ml, and Friction Coefficient of from about 1.2 to about 2.0.

### CROSS REFERENCE TO RELATED APPLICATIONS

This application claims the benefit of U.S. Provisional Application No. 60/794,894, filed Apr. 25, 2006.

### FIELD OF THE INVENTION

The present invention relates to a leave-on companion animal composition comprising; a composition having a Total Lather Volume of from about 300 ml to about 700 ml, IL-1α of from about 0 pg/ml to about 7000 pg/ml, and Friction Coefficient of from about 1.2 to about 2.0.

### BACKGROUND OF THE INVENTION

Grooming a companion animal is necessary in order to maintain the companion animal's health. Typically, the products used to clean, condition, and treat a companion animal must meet certain criteria. These criteria include cleansing effectiveness, skin feel, mildness to skin, hair, and ocular mucosae, pleasant smell, and lather volume. Ideal companion animal cleansers should gently cleanse the skin or hair, causes little or no irritation, and should not leave the skin or hair overly dry after use.

Companion animals tend to squirm and attempt to escape during the cleansing, treatment and conditioning process which results in inefficient use of the cleansing/conditioning product and/or contact with the companion animal. Additionally, with a companion animal the bathing process is normally highly unpleasant for the companion animal and results in increased anxiety, nervousness and as a result of this, an unpleasant experience for the companion animal owner.

Most cleansing compositions for companion animals require a water source to remove the cleansing composition. If the composition is not completely removed, the companion animal may show signs of irritation and itching which may result in a hot spot on the companion animal's skin. Additionally, locating an appropriate water source can be inconvenient or impossible under certain traveling conditions, time constraints and/or by the mere size of the companion animal.

Therefore, the need remains for a composition which is easy to use, suitable for use by consumers, able to cleanse, treat and condition effectively, mild to the skin, hair, and ocular mucosae of the companion animal, provide a pleasant smell, and ideal lather volume. Furthermore, the need remains for a composition that can be applied to the companion animal with out the need to remove with water such that the consumer can focus on the task at hand, namely washing, cleaning, treating, and conditioning and at the same time provide a soothing contact between the user and the companion animal without having to be concerned with attempting to wash the composition off of the companion animal with a source of water.

### SUMMARY OF THE INVENTION

The present invention relates to a leave on companion animal composition comprising; a composition having a Total Lather Volume of from about 300 ml to about 700 ml.

The present invention further relates to a leave on companion animal composition comprising; a composition having an IL-1α of from about 0 pg/ml to about 7000 pg/ml.

The present invention further relates to a leave on companion animal composition comprising; a composition having a Total Lather Volume of from about 300 ml to about 700 ml; said composition having IL-1α of from about 0 pg/ml to about 7000 pg/ml; and said composition having a Friction Coefficient of from about 1.2 to about 2.0.

The present invention further relates to an article of commerce comprising: a container comprising; a leave on companion animal composition, which provides conditioning, cleansing and treatment benefits to a companion animal when applied and comprises: (a) a composition having a Total Lather Volume from about 300 ml to about 700 ml; (b) said composition having IL-1α of from about 0 pg/ml to about 7000 pg/ml; wherein said container has instructions for conditioning, cleansing, and treating the companion animal, comprising the instructions to wash as normal, massage said companion animal, and optionally pat dry with a towel.

The present invention further relates methods for cleansing, conditioning, and treating the skin or hair and similar keratin-containing surfaces of the companion animal, primarily skin and hair using the leave-on composition described herein.

The present invention further relates to a kit for a companion animal comprising: (a) a composition; and (b) a container.

### DETAILED DESCRIPTION OF THE INVENTION

The instant leave on composition, and methods of the present invention, is suitable for use by a user, in cleansing, treating, and conditioning a companion animal. Due to the ease and simple method of use a user is able to clean, treat, or condition their companion animal, with the instant invention.

As used herein, the term “companion animal” means an animal including (for example) dogs, cats, horses, rabbits, guinea pig, hamster, gerbil, ferret, zoo mammals and the like. Dogs, rabbits, horses and cats are particularly preferred.

By “leave-on” as used herein, refers to compositions that can be both rinsed off the companion animal after application or left on depending upon the desire of the user. Preferably the composition is left on.

The term “fluid” is used herein to mean “fluids” selected from the group consisting of water, mono- and polyhydric alcohols (glycerin, propylene glycol, ethanol, isopropanol, etc.), hydrocarbon oils such as mineral oil, silicone fluids, also triglyceride oils, also fluid resins such as silicone MQ resins, esters and ethers of hydrocarbons, alcohols, perfume, fragrance oils, natural oils such as terpenes, various tree and plant oils, as well as mixtures of the above and can contain other components dissolved or dispersed within them, or in addition to them.

The phrase “substantially free of” as used herein, means that the composition comprises less than about 3%, preferably less than about 1%, more preferably less than about 0.5%, even more preferably less than about 0.25%, and still more preferably less than about 0.1%, even still more preferably less than 0.01% by weight of the composition, of the stated ingredient.

Composition

The present invention is a leave on composition used by individuals preferably for cleansing, conditioning and or treatment of skin, hair, nails, ears, paws or other similar keratin-containing surfaces of a companion animal. The leave on composition can be used on all regions of the companion animal. The leave on composition of the present invention can be liquid or semi-liquid, cream or mousse composition. The product forms contemplated for purposes of defining the compositions and methods of the present invention are typically leave on compositions, by which is meant the composition is applied topically to the companion animal and then subsequently (i.e., within minutes) left on, and/or rinsed away with water, and/or otherwise wiped off using a substrate or other suitable removal means. Additionally, the composition can be associated with an implement such as a mitt, mitten and/or glove and then topically applied to the companion animal.

Preferably, the leave on compositions are mild, which means that these composition provides sufficient cleansing or detersive benefits but do not overly dry the companion animal, and yet meet the lathering criteria such that the agents do not generate a substantial lather.

The leave on composition of the present invention having a Total Lather Volume from about 300 ml to about 700 ml, from about 310 ml to about 600 ml, from about 315 ml to about 500 ml, from about 320 ml to about 400 ml, and from about 325 ml to about 380 ml as measured by the Lather Volume Test described hereafter.

The leave on composition of the present invention having a Flash Lather Volume from about 100 ml to about 230 ml, from about 105 ml to about 200 ml, from about 110 ml to about 180 ml, from about 120 ml to about 170 ml, and from about 125 ml to about 160 ml as measured by the Lather Volume Test described hereafter.

Viability of the skin and inflammatory cytokine release are well-established measures that correlate to adverse skin effects, such as irritation. The leave on composition of the present invention having an IL1α of from about 0 picograms/milliliter (pg/ml) to about 7000 pg/ml, from about 70 pg/ml to about 6000 pg/ml, from about 90 pg/ml to about 5000 pg/ml, from about 100 pg/ml to about 4000 pg/ml, from about 110 pg/ml to about 3000 pg/ml as measured by the Irritation Assessment Test described hereafter.

The leave on composition of the present invention having a Viability from about 20% to about 100%, from about 25% to about 90%, from about 30% to about 85% to about 35% to about 80%, and from about 40% to about 75%, as measured by the Irritation Assessment Test described hereafter.

The leave on composition of the present invention having a IL8 from about 0 to about 8 picograms/ml (pg/ml) to about 800 pg/ml, from about 50 pg/ml to about 700 pg/ml, from about 90 pg/ml to about 600 pg/ml, from about 100 pg/ml to about 500 pg/ml, from about 150 pg/ml to about 400 pg/ml, and from about 200 pg/ml to about 300 pg/ml, as measured by the Irritation Assessment Test described hereafter.

After treatment with the leave on compositions of the present invention, hair will preferably demonstrate a Friction Coefficient of from about 1.2 to about 2, from about 1.3 to about 1.9, from about 1.4 to about 1.8, from about 1.5 to about 1.8. The Friction Coefficient is determined according to the Friction Test described hereafter.

Surfactant Component

The compositions of the present invention can comprise a surfactant component. The surfactant component comprises surfactants suitable for application to the companion animal. The surfactant is selected from the group consisting of anionic surfactant, non-ionic surfactant, zwitterionic surfactant, cationic surfactant, soap, and mixtures thereof.

When present, the leave on composition comprises a surfactant component at concentrations ranging from about 0.1% to about 95%, from about 0.5% to about 95%, from about 1% to about 90%, from about 5% to about 80%, from about 10% to about 70%, and from about 15% to about 60%, by weight of the composition. The surfactant component comprises the surfactant at concentrations ranging from about 0.1% to about 50%, from about 1% to about 35%, from about 5% to about 30%, from about 8% to about 25%, and from about 10% to about 24%, by weight of the surfactant component. The preferred pH range of the leave on composition is from about 4 to about 9, more preferably about 7.

Anionic Surfactants

The leave on composition can comprise an anionic surfactant at concentrations ranging from about 0.1% to about 50%, from about 0.4% to about 30%, from about 0.5% to about 25%, from about 1% to about 20%, from about 2% to about 10%, by weight of the surfactant component.

Non-limiting examples of anionic surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678, to Laughlin et al., issued Dec. 30, 1975.

A wide variety of anionic surfactants are useful herein. Nonlimiting examples of anionic surfactants include those selected from the group consisting of alkyl and alkyl ether sulfates, sulfated monoglycerides, sulfonated olefins, alkyl aryl sulfonates, primary or secondary alkane sulfonates, alkyl sulfosuccinates, acyl taurates, acyl isethionates, alkyl glycerylether sulfonate, sulfonated methyl esters, sulfonated fatty acids, alkyl phosphates, acyl glutamates, acyl sarcosinates, alkyl sulfoacetates, acylated peptides, alkyl ether carboxylates, acyl lactylates, anionic fluorosurfactants, and combinations thereof.

Non-limiting examples of anionic surfactants include those selected from the group consisting of sarcosinates, sulfates, ethoxylated sulfate, sulfonates, glyceryl sulfonates, isethionates, phosphates, taurates, lactylates, glutamates, soaps, sulfosuccinates, ethoxylated sulfosuccinates, and mixtures thereof.

Other anionic materials useful herein include are fatty acid soaps (i.e., alkali metal salts, e.g., sodium or potassium salts) typically having from a fatty acid having about 8 to about 24 carbon atoms, preferably from about 10 to about 20 carbon atoms. These fatty acids used in making the soaps can be obtained from natural sources such as, for instance, plant or animal-derived glycerides (e.g., palm oil, coconut oil, soybean oil, castor oil, tallow, lard, etc.) Additionally, anionic materials include natural soaps derived from the saponification of vegetable and/or animal fats & oils examples of which include sodium laurate, sodium myristate, palmitate, stearate, tallowate, cocoate. The fatty acids can also be synthetically prepared. Soaps and their preparation are described in detail in U.S. Pat. No. 4,557,853.

Anionic surfactants for use in the composition include alkyl and alkyl ether sulfates. These materials have the respective formulae R1O—SO3M and R1(CH2H4O)x -O—SO3M, wherein R1 is a saturated or unsaturated, branched or unbranched alkyl group from about 8 to about 24 carbon atoms, x is 1 to 10, and M is a water-soluble cation such as ammonium, sodium, potassium, magnesium, triethanolamine, diethanolamine and monoethanolamine. The alkyl sulfates are typically made by the sulfation of monohydric alcohols (having from about 8 to about 24 carbon atoms) using sulfur trioxide or other known sulfation technique. The alkyl ether sulfates are typically made as condensation products of ethylene oxide and monohydric alcohols (having from about 8 to about 24 carbon atoms) and then sulfated. These alcohols can be derived from fats, e.g., coconut oil or tallow, or can be synthetic. Specific examples of alkyl sulfates which may be used in the composition are sodium, ammonium, potassium, magnesium, or TEA salts of lauryl or myristyl sulfate. Examples of alkyl ether sulfates which may be used include ammonium, sodium, magnesium, or TEA laureth-3 sulfate.

Another suitable class of anionic surfactants are the sulfated monoglycerides of the form R1 CO—O—CH2-C(OH)H—CH2-O—SO3M, wherein R1 is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These are typically made by the reaction of glycerin with fatty acids (having from about 8 to about 24 carbon atoms) to form a monoglyceride and the subsequent sulfation of this monoglyceride with sulfur trioxide. An example of a sulfated monoglyceride is sodium cocomonoglyceride sulfate.

Other suitable anionic surfactants include olefin sulfonates of the form R1SO3M, wherein R1 is a mono-olefin having from about 12 to about 24 carbon atoms, and M is a water-soluble cation such as ammonium, sodium, potassium, magnesium, triethanolamine, diethanolamine and monoethanolamine. These compounds can be produced by the sulfonation of alpha olefins by means of uncomplexed sulfur trioxide, followed by neutralization of the acid reaction mixture in conditions such that any sultones which have been formed in the reaction are hydrolyzed to give the corresponding hydroxyalkanesulfonate. An example of a sulfonated olefin is sodium C14/C16 alpha olefin sulfonate.

Other suitable anionic surfactants are the linear alkylbenzene sulfonates of the form R1-C₆H₄—SO3M, wherein R1 is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These are formed by the sulfonation of linear alkyl benzene with sulfur trioxide. An example of this anionic surfactant is sodium dodecylbenzene sulfonate.

Still other anionic surfactants suitable for this composition include the primary or secondary alkane sulfonates of the form R1SO3M, wherein R1 is a saturated or unsaturated, branched or unbranched alkyl chain from about 8 to about 24 carbon atoms, and M is a water-soluble cation such as ammonium, sodium, potassium, magnesium, triethanolamine, diethanolamine and monoethanolamine. These are commonly formed by the sulfonation of paraffins using sulfur dioxide in the presence of chlorine and ultraviolet light or another known sulfonation method. The sulfonation can occur in either the secondary or primary positions of the alkyl chain. An example of an alkane sulfonate useful herein is alkali metal or ammonium C13-C17 paraffin sulfonates.

Still other suitable anionic surfactants are the alkyl sulfosuccinates, which include disodium N-octadecylsulfosuccinamate; diammonium lauryl sulfosuccinate; tetrasodium N-(1,2-dicarboxyethyl)-N-octadecylsulfosuccinate; diamyl ester of sodium sulfosuccinic acid; dihexyl ester of sodium sulfosuccinic acid; and dioctyl esters of sodium sulfosuccinic acid.

Also useful are taurates which are based on taurine, which is also known as 2-aminoethanesulfonic acid. Examples of taurates include N-alkyltaurines such as the one prepared by reacting dodecylamine with sodium isethionate as detailed in U.S. Pat. No. 2,658,072 which is incorporated herein by reference in its entirety. Other examples based of taurine include the acyl taurines formed by the reaction of n-methyl taurine with fatty acids (having from about 8 to about 24 carbon atoms).

Another class of anionic surfactants suitable for use in the composition is the acyl isethionates. The acyl isethionates typically have the formula R1CO—O—CH2CH2SO3M wherein R1 is a saturated or unsaturated, branched or unbranched alkyl group having from about 10 to about 30 carbon atoms, and M is a cation. These are typically formed by the reaction of fatty acids (having from about 8 to about 30 carbon atoms) with an alkali metal isethionate. Nonlimiting examples of these acyl isethionates include ammonium cocoyl isethionate, sodium cocoyl isethionate, sodium lauroyl isethionate, and mixtures thereof.

Still other suitable anionic surfactants are the alkylglyceryl ether sulfonates of the form R1-OCH2-C(OH)H—CH2-SO3M, wherein R1 is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These can be formed by the reaction of epichlorohydrin and sodium bisulfite with fatty alcohols (having from about 8 to about 24 carbon atoms) or other known methods. One example is sodium cocoglyceryl ether sulfonate.

Other suitable anionic surfactants include the sulfonated fatty acids of the form R1-CH(SO4)-COOH and sulfonated methyl esters of the from R1-CH(SO4)-CO—O—CH3, where R1 is a saturated or unsaturated, branched or unbranched alkyl group from about 8 to about 24 carbon atoms. These can be formed by the sulfonation of fatty acids or alkyl methyl esters (having from about 8 to about 24 carbon atoms) with sulfur trioxide or by another known sulfonation technique. Examples include alpha sulphonated coconut fatty acid and lauryl methyl ester.

Other anionic materials include phosphates such as monoalkyl, dialkyl, and trialkylphosphate salts formed by the reaction of phosphorous pentoxide with monohydric branched or unbranched alcohols having from about 8 to about 24 carbon atoms. These could also be formed by other known phosphation methods. An example from this class of surfactants is sodium mono or dilaurylphosphate.

Other anionic materials include acyl glutamates corresponding to the formula R1CO—N(COOH)—CH2CH2-CO2M wherein R1 is a saturated or unsaturated, branched or unbranched alkyl or alkenyl group of about 8 to about 24 carbon atoms, and M is a water-soluble cation. Nonlimiting examples of which include sodium lauroyl glutamate and sodium cocoyl glutamate.

Other anionic materials include alkanoyl sarcosinates corresponding to the formula R1 CON(CH3)-CH2CH2-CO2M wherein R1 is a saturated or unsaturated, branched or unbranched alkyl or alkenyl group of about 10 to about 20 carbon atoms, and M is a water-soluble cation. Nonlimiting examples of which include sodium lauroyl sarcosinate, sodium cocoyl sarcosinate, and ammonium lauroyl sarcosinate.

Other anionic materials include alkyl ether carboxylates corresponding to the formula R1-(OCH2CH2)ₓ—OCH2-CO2M wherein R1 is a saturated or unsaturated, branched or unbranched alkyl or alkenyl group of about 8 to about 24 carbon atoms, x is 1 to 10, and M is a water-soluble cation. Nonlimiting examples of which include sodium laureth carboxylate.

Other anionic materials include acyl lactylates corresponding to the formula R1CO—[O—CH(CH3)-CO]x-CO2M wherein R1 is a saturated or unsaturated, branched or unbranched alkyl or alkenyl group of about 8 to about 24 carbon atoms, x is 3, and M is a water-soluble cation. Nonlimiting examples of which include sodium cocoyl lactylate.

Other anionic materials include the carboxylates, nonlimiting examples of which include sodium lauroyl carboxylate, sodium cocoyl carboxylate, and ammonium lauroyl carboxylate. Anionic flourosurfactants can also be used.

Other anionic materials include phosphates such as monoalkyl, dialkyl, and trialkylphosphate salts. Non-limiting examples of preferred anionic surfactants useful herein include those selected from the group consisting of sodium lauryl sulfate, ammonium lauryl sulfate, ammonium laureth sulfate, sodium laureth sulfate, sodium trideceth sulfate, ammonium cetyl sulfate, sodium cetyl sulfate, ammonium cocoyl isethionate, sodium lauroyl isethionate, sodium lauroyl lactylate, triethanolamine lauroyl lactylate, sodium caproyl lactylate, sodium lauroyl sarcosinate, sodium myristoyl sarcosinate, sodium cocoyl sarcosinate, sodium lauroyl methyl taurate, sodium cocoyl methyl taurate, sodium lauroyl glutamate, sodium myristoyl glutamate, and sodium cocoyl glutamate and mixtures thereof.

Non-Ionic Surfactants

The leave on composition can comprise a nonionic surfactant at concentrations ranging from about 0.1% to about 50%, from about 0.25% to about 30%, from about 0.5% to about 25%, from about 1.0% to about 20%, and from about 1.5% to about 10%, by weight of the surfactant component.

Non-limiting examples of nonionic surfactants for use in the compositions of the present invention are disclosed in McCutcheon's, Detergents and Emulsifiers, North American edition (1986), published by allured Publishing Corporation; and McCutcheon's, Functional Materials, North American Edition (1992).

Nonionic surfactants useful herein include those selected from the group consisting of alkyl glucosides, polyglucosides, polyhydroxy fatty acid amides, alkoxylated fatty acid esters, sugar esters, ethoxylated esters, glycerol esters, ethoxylates, propoxylates, PEG/PPG copolymers, glycerides, sorbitans, and mixtures. More specifically, polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, and mixtures.

Alkyl glucosides and alkyl polyglucosides are useful herein, and can be broadly defined as condensation products of long chain alcohols, e.g., C8-30 alcohols, with sugars or starches or sugar or starch polymers, i.e., glycosides or polyglycosides. These compounds can be represented by the formula (S)ₙ—O—R wherein S is a sugar moiety such as glucose, fructose, mannose, and galactose; n is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C8-20 alkyl group, and n is an integer of from about 1 to about 9. Commercially available examples of these surfactants include decyl polyglucoside (available as APG 325 CS from Henkel) and lauryl polyglucoside (available as APG 600CS and 625 CS from Henkel). Also useful are sucrose ester surfactants such as sucrose cocoate and sucrose laurate.

Other useful nonionic surfactants include polyhydroxy fatty acid amide surfactants, more specific examples of which include glucosamides, corresponding to the structural formula:

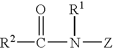

wherein: R¹ is H, C₁-C₄ alkyl, 2-hydroxyethyl, 2-hydroxy- propyl, preferably C₁-C₄ alkyl, more preferably methyl or ethyl, most preferably methyl; R² is C₅-C₃₁ alkyl or alkenyl, preferably C₇-C₁₉ alkyl or alkenyl, more preferably C₉-C₁₇ alkyl or alkenyl, most preferably C₁₁-C₁₅ alkyl or alkenyl; and Z is a polhydroxyhydrocarbyl moiety having a linear hydrocarbyl chain with a least 3 hydroxyls directly connected to the chain, or an alkoxylated derivative (preferably ethoxylated or propoxylated) thereof. Z preferably is a sugar moiety selected from the group consisting of glucose, fructose, maltose, lactose, galactose, mannose, xylose, and mixtures thereof. An especially preferred surfactant corresponding to the above structure is coconut alkyl N-methyl glucoside amide (i.e., wherein the R²CO— moiety is derived from coconut oil fatty acids). Processes for making compositions containing polyhydroxy fatty acid amides are disclosed, for example, in G.B. Patent Specification 809,060, published Feb. 18, 1959, by Thomas Hedley & Co., Ltd.; U.S. Pat. No. 2,965,576, to E. R. Wilson, issued Dec. 20, 1960; U.S. Pat. No. 2,703,798, to A. M. Schwartz, issued Mar. 8, 1955; and U.S. Pat. No. 1,985,424, to Piggott, issued Dec. 25, 1934; each of which are incorporated herein by reference in their entirety.

Other examples of nonionic surfactants include amine oxides. Amine oxides correspond to the general formula R₁R₂R₃N→O, wherein R₁ contains an alkyl, alkenyl or monohydroxy alkyl radical of from about 8 to about 18 carbon atoms, from 0 to about 10 ethylene oxide moieties, and from 0 to about 1 glyceryl moiety, and R₂ and R₃ contain from about 1 to about 3 carbon atoms and from 0 to about 1 hydroxy group, e.g., methyl, ethyl, propyl, hydroxyethyl, or hydroxypropyl radicals. The arrow in the formula is a conventional representation of a semipolar bond. Examples of amine oxides suitable for use in this invention include dimethyl-dodecylamine oxide, oleyldi(2-hydroxyethyl) amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Nonlimiting examples of nonionic surfactants for use herein are those selected form the group consisting of C₈-C₁₄ glucose amides, C₈-C₁₄ alkyl polyglucosides, sucrose cocoate, sucrose laurate, lauramine oxide, cocoamine oxide, and mixtures thereof.

Amphoteric Surfactants

The leave on composition can comprise an amphoteric surfactant at concentrations ranging from about 0.1% to about 50%, from about 0.4% to about 30%, from about 0.5% to about 25%, from about 1% to about 20%, from about 2% to about 10%, by weight of the surfactant component.

The term “amphoteric surfactant,” as used herein, is also intended to encompass zwitterionic surfactants, which are well known to formulators skilled in the art as a subset of amphoteric surfactants.

A wide variety of amphoteric surfactants can be used in the compositions of the present invention. Particularly useful are those which are broadly described as derivatives of aliphatic secondary and tertiary amines, preferably wherein the nitrogen is in a cationic state, in which the aliphatic radicals can be straight or branched chain and wherein one of the radicals contains an ionizable water solubilizing group, e.g., carboxy, sulfonate, sulfate, phosphate, or phosphonate.

Nonlimiting examples of amphoteric surfactants useful in the compositions of the present invention are disclosed in McCutcheon's, Detergents and Emulsifiers, North American edition (1986), published by allured Publishing Corporation; and McCutcheon's, Functional Materials, North American Edition (1992); both of which are incorporated by reference herein in their entirety.

Nonlimiting examples of amphoteric or zwitterionic surfactants are those selected from the group consisting of amine oxides, betaines, sultaines, hydroxysultaines, alkyliminoacetates, iminodialkanoates, aminoalkanoates, and mixtures thereof.

Examples of betaines include the higher alkyl betaines, such as coco dimethyl carboxymethyl betaine, lauryl dimethyl carboxymethyl betaine, lauryl dimethyl alpha-carboxyethyl betaine, cetyl dimethyl carboxymethyl betaine, cetyl dimethyl betaine (available as Lonzaine 16SP from Lonza Corp.), lauryl bis-(2-hydroxyethyl) carboxymethyl betaine, oleyl dimethyl gamma-carboxypropyl betaine, lauryl bis-(2-hydroxypropyl)alpha-carboxyethyl betaine, coco dimethyl sulfopropyl betaine, lauryl dimethyl sulfoethyl betaine, lauryl bis-(2-hydroxyethyl) sulfopropyl betaine, amidobetaines and amidosulfobetaines (wherein the RCONH(CH₂)₃ radical is attached to the nitrogen atom of the betaine), oleyl betaine (available as amphoteric Velvetex OLB-50 from Henkel), and cocamidopropyl betaine (available as Velvetex BK-35 and BA-35 from Henkel).

Examples of sultaines and hydroxysultaines include materials such as cocamidopropyl hydroxysultaine (available as Mirataine CBS from Rhone-Poulenc).

Preferred for use herein are amphoteric surfactants having the following structure:

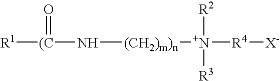

wherein R¹ is unsubstituted, saturated or unsaturated, straight or branched chain alkyl having from about 9 to about 22 carbon atoms. Preferred R¹ has from about 11 to about 18 carbon atoms; more preferably from about 12 to about 18 carbon atoms; more preferably still from about 14 to about 18 carbon atoms; m is an integer from 1 to about 3, more preferably from about 2 to about 3, and more preferably about 3; n is either 0 or 1, preferably 1; R² and R³ are independently selected from the group consisting of alkyl having from 1 to about 3 carbon atoms, unsubstituted or mono-substituted with hydroxy, preferred R² and R³ are CH₃; X is selected from the group consisting of CO₂, SO₃ and SO₄; R⁴ is selected from the group consisting of saturated or unsaturated, straight or branched chain alkyl, unsubstituted or monosubstituted with hydroxy, having from 1 to about 5 carbon atoms. When X is CO₂, R⁴ preferably has 1 or 3 carbon atoms, more preferably 1 carbon atom. When X is SO₃ or SO₄, R⁴ preferably has from about 2 to about 4 carbon atoms, more preferably 3 carbon atoms.

Examples of amphoteric surfactants of the present invention include the following compounds:

Cetyl dimethyl betaine (this material also has the CTFA designation cetyl betaine)

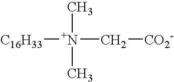

Cocamidopropylbetaine

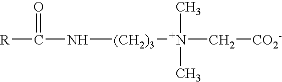

wherein R has from about 9 to about 13 carbon atoms

Cocamidopropyl hydroxy sultaine

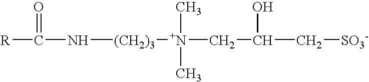

wherein R has from about 9 to about 13 carbon atoms,

Examples of other useful amphoteric surfactants are alkyliminoacetates, and iminodialkanoates and aminoalkanoates of the formulas RN[CH₂)ₘCO₂M]₂ and RNH(CH₂)ₘCO₂M wherein m is from 1 to 4, R is a C₈-C₂₂ alkyl or alkenyl, and M is H, alkali metal, alkaline earth metal ammonium, or alkanolammonium. Also included are imidazolinium and ammonium derivatives. Specific examples of suitable amphoteric surfactants include sodium 3-dodecyl-aminopropionate, sodium 3-dodecylaminopropane sulfonate, N-higher alkyl aspartic acids such as those produced according to the teaching of U.S. Pat. No. 2,438,091 which is incorporated herein by reference in its entirety; and the products sold under the trade name “Miranol” and described in U.S. Pat. No. 2,528,378, which is incorporated herein by reference in its entirety. Other examples of useful amphoterics include amphoteric phosphates, such as coamidopropyl PG-dimonium chloride phosphate (commercially available as Monaquat PTC, from Mona Corp.). Also useful are amphoacetates such as disodium lauroamphodiacetate, sodium lauroamphoacetate, and mixtures thereof.

Amphoacetates and diamphoacetates may also be used.

### Amphoacetate

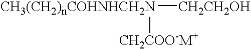

### Diamphoacetate

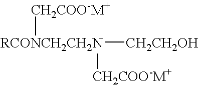

Amphoacetates and diamphoacetates conform to the formulas (above) where R is an aliphatic group of 8 to 18 carbon atoms. M is a cation such as sodium, potassium, ammonium, or substituted ammonium. Sodium lauroamphoacetate, sodium cocoamphoactetate, disodium lauroamphoacetate, and disodium cocodiamphoacetate are preferred in some embodiments.

The composition may further comprise at least one zwitterionic surfactant. The composition comprises an zwitterionic surfactant at concentrations ranging from about 0.1% to about 50%, from about 0.4% to about 30%, from about 0.5% to about 25%, from about 1% to about 20%, from about 2% to about 10%, by weight of the surfactant component.

Zwitterionic surfactants suitable for use in the compositions include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula:

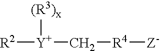

wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants suitable for use in the compositions include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

Cationic surfactants can also be used in the compositions, but are generally less preferred, and preferably represent less than about 5% by weight of the compositions.

### Conditioning Agents

The compositions of the present invention can comprise a conditioning agent that is useful for providing a conditioning benefit to the skin, hair and other parts of the companion animal's body. The conditioning agents can be alone or in combination with the surfactant component, and/or treatment agents. The leave on composition can comprise conditioning agents at concentrations ranging from about 0.1% to about 95%, from about 0.5% to about 90%, from about 1% to about 85%, from about 5% to about 70%, from about 10% to about 60%, and from about 15% to about 50%, from about 18% to about 30%, from about 20% to about 25%, by weight of the composition.

The conditioning agent useful in the present invention can comprise: a water soluble conditioning agent; an oil soluble conditioning agent; a conditioning emulsion; or any combination or permutation of the three. The oil soluble conditioning agent is selected from one or more oil soluble conditioning agents such that the weighted arithmetic mean solubility parameter of the oil soluble conditioning agent is less than or equal to 10.5. The water soluble conditioning agent is selected from one or more water soluble conditioning agents such that the weighted arithmetic mean solubility parameter of the water soluble conditioning agent is greater than 10.5. It is recognized, based on this mathematical definition of solubility parameters, that it is possible, for example, to achieve the required weighted arithmetic mean solubility parameter, i.e. less than or equal to 10.5, for an oil soluble conditioning agent comprising two or more compounds if one of the compounds has an individual solubility parameter greater than 10.5. Conversely, it is possible to achieve the appropriate weighted arithmetic mean solubility parameter, i.e. greater than 10.5, for a water soluble conditioning agent comprising two or more compounds if one of the compounds has an individual solubility parameter less than or equal to 10.5.

Solubility parameters are well known to the formulation chemist of ordinary skill in the art and are routinely used as a guide for determining compatibilities and solubilities of materials in the formulation process. See “Solubility Effects in Product, Package, Penetration, and Preservation”, Cosmetics and Toiletries vol. 103, p 47-69, (October 1988).

Non-limiting examples of useful conditioning agents include those selected from the group consisting of silicone, functional silicone polymers, functional silicone polymers, fatty acids, esters of fatty acids, fatty alcohols, ethoxylates, polyol polyesters, glycerine, glycerin mono-esters, glycerin polyesters, epidermal and sebaceous hydrocarbons, lanolin, straight and branched hydrocarbons, silicone oil, silicone gum, vegetable oil, vegetable oil adduct, hydrogenated vegetable oils, nonionic polymers, natural waxes, petrolatum, petrolatum derivatives, synthetic waxes, polyolefinic glycols, polyolefinic monoester, polyolefinic polyesters, cholesterols, cholesterol esters, triglycerides and mixtures thereof.

More particularly, the conditioning agent may be selected from the group consisting of silicone polymers, functional silicone polymers, paraffin, mineral oil, petrolatum, stearyl alcohol, cetyl alchohol, cetearyl alcohol, behenyl alcohol, C10-30 polyesters of sucrose, stearic acid, palmitic acid, behenic acid, oleic acid, linoleic acid, myristic acid, lauric acid, ricinoleic acid, steareth-1-100, cetereath 1-100, cholesterols, cholesterol esters, glyceryl tribehenate, glyceryl dipalmitate, glyceryl monostearate, trihydroxystearin, ozokerite wax, jojoba wax, lanolin wax, ethylene glycol distearate, candelilla wax, carnauba wax, beeswax, and silicone waxes.

Mineral oil, which is also known as petrolatum liquid, is a mixture of liquid hydrocarbons obtained from petroleum. See The Merck Index, Tenth Edition, Entry 7048, p. 1033 (1983) and International Cosmetic Ingredient Dictionary, Fifth Edition, vol. 1, p. 415-417 (1993).

Petrolatum, which is also known as petroleum jelly, is a colloidal system comprising nonstraight-chain solid hydrocarbons and high-boiling liquid hydrocarbons. See The Merck Index, Tenth Edition, Entry 7047, p. 1033 (1983); Schindler, Drug. Cosmet. Ind., p. 89, 36-37, 76, 78-80, 82 (1961); and International Cosmetic Ingredient Dictionary, Fifth Edition, Vol. 1, p. 537 (1993).

Nonvolatile silicones such as polydialkylsiloxanes, polydiarylsiloxanes, and polyalkarylsiloxanes are also useful skin conditioning agents. These silicones are disclosed in U.S. Pat. No. 5,069,897, to Orr, issued Dec. 3, 1991.

The conditioning agent preferably used in the present invention may also comprise a conditioning emulsion that is useful for providing a conditioning benefit to the skin, hair, paws and nails during the use of the leave-on composition. The term “conditioning emulsion” as used herein can either mean the combination of an internal phase comprising a water soluble conditioning agent that is enveloped by an external phase comprising an oil soluble agent or the term “conditioning emulsion” as used herein means the combination of an internal phase comprising an oil soluble agent that is enveloped by an external phase comprising a water soluble agent. In preferred embodiments, the conditioning emulsion would further comprise an emulsifier. The conditioning emulsion comprises from about from about 0.01% to about 100%, from about 0.1% to about 95%, from about 1% to about 90%, 1.5% to about 85%, from about 2% to about 80%, from about 5% to about 70%, and from about 10% to about 50% by weight of said leave-on composition. In a preferred embodiment the conditioning emulsion comprises (i) an internal phase comprising water soluble conditioning agents as described above, and (ii) an external phase comprising oil soluble agents as described hereinbefore in the oil soluble conditioning agent section or hereinafter in the “Materials Used to Increase Lipid Hardness Value” section. In further embodiments, the conditioning emulsion further comprises an emulsifier capable of forming an emulsion of said internal and external phases. Although an emulsifier capable of forming an emulsion of the internal and external phases is preferred in the present invention, it is recognized in the art of skin care formulations that a water soluble conditioning agent can be enveloped by an oil soluble agent without an emulsifier.

Treatment Agents

The compositions of the present invention can comprise a treatment agent that is useful for providing a therapeutic benefit and/or cosmetic benefit to the skin, hair, paws, ears, nails and similar keratin-containing surfaces of the companion animal during the use of the leave-on composition. The treatment agents are suitable for application to keratin-containing tissue, that is, they are suitable for use in contact with companion animal without undue toxicity, incompatibility, instability, allergic response, and the like.

The treatment agents useful in the present invention can comprise compositions comprising the following nonlimiting examples, vitamins, cylodextrins, zeolites, peptides, sunscreen actives, terpene alcohols, desquamation actives including a combination of sulfhydryl compounds and zwitterionic surfactants, and a combination of salicylic acid and zwitterionic surfactants, anti-atrophy actives, anti-oxidants/radical scavengers, flavonoids, anti-inflammatory agent, topical anesthetics, chelators, antimicrobial and antifungal actives, skin soothing and skin healing actives, flea actives, moisturizing actives, tick actives, other insect active, and mixtures thereof.

The leave on composition can comprise treatment agents at concentrations ranging from about 0.1% to about 95%, from about 0.5% to about 90%, from about 1% to about 85%, from about 5% to about 70%, from about 10% to about 60%, and from about 15% to about 50%, from about 18% to about 30%, from about 20% to about 25%, by weight of the composition.

Foam Agent

The leave on composition of the present invention can comprise a foam agent. Foam agents can be used to aid in controlling the lather volume generated by the composition of the present invention. The leave on composition can comprise foam agents at concentrations ranging from about 0.01% to about 2%, from about 0.05% to about 1.5%, from about 0.1% to about 1%, from about 0.15% to about 0.7%, from about 0.2% to about 0.5%, and from about 0.25% to about 0.4%, by weight of the composition.

Nonlimiting examples include silicones, fatty acids, alcohols, hydrophobes and mixtures thereof.

Additional nonlimiting examples include the following: alcohol, bisphenylhexamethicone, cetyl dimethicone, dimethicone, dimethicone silyate, dimethiconol, diphenyl dimethicone, hexadecyl methicone, hexamethyldisiloxane, hexyl alcohol, isopropyl alcohol, petroleum distillates, phenyl disiloxane, phenyldimethicone, phenyltrimethicone, polydimethylsiloxane, propyl alcohol, silica silylate, simethicone, trimethylsiloxysilicate, triphenyl trrimethicone and mixtures thereof.

Additional Ingredients

The compositions of the present invention can comprise a wide range of other optional components. These additional components should be pharmaceutically acceptable. The CTFA Cosmetic Ingredient Handbook, Second Edition, 1992, describes a wide variety of nonlimiting cosmetic and pharmaceutical ingredients commonly used in the skin care industry, which are suitable for use in the compositions of the present invention. Nonlimiting examples of functional classes of ingredients are described at page 537 of this reference. Examples of these and other functional classes include: abrasives, absorbents, antioxidants, binders, biological additives, buffering agents, bulking agents, chemical additives, colorants, cosmetic biocides, denaturants, drug astringents, external analgesics, film formers, fragrance components, humectants, opacifying agents, pH adjusters, preservatives, propellants, reducing agents, and skin bleaching agents.

Also useful herein are aesthetic components such as fragrances, pigments, colorings, essential oils, skin sensates, astringents, skin soothing agents, and skin healing agents.

The compositions used in the present invention may also contain a “fluid” such as water, mono- and polyhydric alcohols (glycerin, propylene glycol, ethanol, isopropanol, etc.), hydrocarbon oils such as mineral oil, silicone oils having a viscosity, and can contain other components dissolved or dispersed within them, or in addition to them.

The present invention also covers kits comprising a leave-on composition and a container. In addition, a kit for a companion animal composition comprising: (a) a leave-on composition comprising; a composition having a Total Lather Volume of from about 300 ml to about 700 ml; and (b) a container with said composition comprises within.

### Method of Use

The leave-on compositions of the present invention are preferably applied topically to the desired area of the skin, hair, paws, ears, nails and similar keratin-containing surfaces of the companion animal in an amount sufficient to provide effective delivery of the leave-on composition. The compositions can be applied directly to the skin or indirectly via the use of a disposable nonwoven implement, cleansing puff, washcloth, sponge or other implement.

The present invention is therefore also directed to methods of cleansing, conditioning and/or treating skin, hair, paws, ears, nails and similar keratin-containing surfaces of the companion animal through the above-described application of the compositions of the present invention.

The present invention is directed to methods of using the leave-on compositions of the present invention comprising the steps of: (A) preparing a leave-on composition having a Total Lather Volume of from about 300 ml to about 700 ml; (B) applying the product of step (A) to a companion animal; and (C) wash the companion animal; (D) Optionally rinse said companion animal; and (E) optionally pat dry with a towel.

While not wishing to be bound by theory, it is believed that efficacy of the product can be linked to the ability of the consumer to understand the usage instructions and to use the product accordingly. The instruction set included may contain pictures or illustrations of the product being applied as well as written instructions. Therefore, the present invention also relates to an article of commerce comprising a container comprising a leave-on composition, which provides conditioning, cleansing, and/or treatment benefits to the companion animal when applied and comprises: (a) a composition having a Total Lather Volume from about 300 ml to about 700 ml; (b) said composition having IL-1α of from about 0 pg/ml to about 7000 pg/ml; wherein said container has instructions for conditioning, cleansing, and treating the companion animal, comprising the instructions to apply the leave-on composition as normal, massage said companion animal; optionally rinse; and optionally pat dry with a towel.

### Method of Manufacture

The leave-on composition of the present invention may be prepared by any known or otherwise effective technique, suitable for making and formulating the desired product form.

Methods

### Lather Volume Test:

Lather volume of a composition, is measured using a graduated cylinder and a rotating apparatus. A 1,000 ml graduated cylinder is used which is marked in 10 ml increments and has a height of 14.5 inches at the 1,000 ml mark from the inside of its base (for example, Pyrex No. 2982). Distilled water (100 grams at 25° C.) is added to the graduated cylinder. The cylinder is clamped in a rotating device, which clamps the cylinder with an axis of rotation that transects the center of the graduated cylinder. Inject 0.50 grams of a composition from a syringe (weigh to ensure proper dosing) into the graduated cylinder onto the side of the cylinder, above the water line, and cap the cylinder. When the sample is evaluated, use only 0.25 cc, keeping everything else the same. The cylinder is rotated for 20 complete revolutions at a rate of about 10 revolutions per 18 seconds, and stopped in a vertical position to complete the first rotation sequence. A timer is set to allow 15 seconds for lather generated to drain. After 15 seconds of such drainage, the first lather volume is measured to the nearest 10 ml mark by recording the lather height in ml up from the base (including any water that has drained to the bottom on top of which the lather is floating).

If the top surface of the lather is uneven, the lowest height at which it is possible to see halfway across the graduated cylinder is the first lather volume (ml). If the lather is so coarse that a single or only a few foam cells which comprise the lather (“bubbles”) reach across the entire cylinder, the height at which at least 10 foam cells are required to fill the space is the first lather volume, also in ml up from the base. Foam cells larger than one inch in any dimension, no matter where they occur, are designated as unfilled air instead of lather. Foam that collects on the top of the graduated cylinder but does not drain is also incorporated in the measurement if the foam on the top is in its own continuous layer, by adding the ml of foam collected there using a ruler to measure thickness of the layer, to the ml of foam measured up from the base. The maximum lather height is 1,000 ml (even if the total lather height exceeds the 1,000 ml mark on the graduated cylinder). 30 seconds after the first rotation is completed, a second rotation sequence is commenced which is identical in speed and duration to the first rotation sequence. The second lather volume is recorded in the same manner as the first, after the same 15 seconds of drainage time. A third sequence is completed and the third lather volume is measured in the same manner, with the same pause between each for drainage and taking the measurement.

The lather results after each sequence are added together and the Total Lather Volume determined as the sum of the three measurements, in milliliters (“ml”). The Flash Lather Volume is the result after the first rotation sequence only, in ml, i.e., the first lather volume. Compositions according to the present invention perform significantly better in this test than similar compositions in conventional emulsion form.

### Friction Test

The method measures the change in friction of an untreated versus treated hair switch with composition. A weighted “sled” (˜47 mm L×36 mm H×35 mm W) weighing 510 g+/−5% and covered with a foam layer on bottom is attached to a force measurement device such as an Instron and pulled at a constant speed across a 10 g hair switch (approximately 6 inches in length) that is clamped on one end and affixed on the other end by tape.

The hair that is used is brown Caucasian hair that has been formed into ˜10 gram switches that are six inches in length which has been cleaned to remove any foreign soils. The hair switches are then allowed to equilibrate in a constant temperature room at 75° C./50% relative humidity (RH) overnight.

The hair switches are treated as follows: 1.0 cc of the composition is then applied to the surface of the hair switch and rubbed into the switch for ˜30-40 sec. Switches are then dried and are re-hung on the rack and placed in a constant temperature room (75° C./50% RH) to equilibrate overnight.

The switch to be tested is clamped into position on a horizontal testing stand and combed 2-3 times to orient the hair and remove tangles. The “sled” is then attached and placed on the hair switch. Friction is measured on switches in the forward direction (toward tip end of hair) by pulling the “sled” at a rate of approximately 1 cm/sec while measuring the tension force (typically measured in grams). Each force measurement is an average of at least ten values recorded over a distance of at least 5 cm once the “sled” has reached constant speed. A minimum of five measurements are taken for each switch. The Friction Coefficient is determined as the average ratio of the friction of the untreated hair switch divided by the friction of the treated hair switch.

### Irritation Assessment Test

Epidermal skin equivalents are multi-layered, differentiated cultures of keratinocytes grown at the air-liquid interface. A stratum corneum is produced naturally upon exposure of the apical surface to air, enabling topical application of complete compositions.

### Procedures:

1. Culture and Treatment of EpiDerm 200 Cultures: EpiDerm 200 cultures (MatTek) are received and are unpacked and placed into 6 well tissue culture plates containing 1 mL/well of assay medium (Mattek, EPI-100 Maintenance/Assay medium, hydrocortisone free). Tissues cultures are equilibrated overnight at 37° C., 5% CO₂, and 90-95% humidity in a Form a tissue culture incubator. The medium is aspirated from the bottom well of each culture and replenished with 900 uL/culture of fresh, prewarmed medium above. Six cultures are dosed topically to determine the most appropriate exposure time for comparison of all the compositions. Upon completion of the pilot study, medium is aspirated from the remaining tissue cultures and replenished with 900 uL fresh, prewarmed assay medium. The tissue cultures are then dosed topically with 40 uL each of the compositions, and replaced into the tissue culture incubator for 26 hours of incubation. After incubation, the assay medium is colleted for IL1α and IL8 testing. The tissue culture is placed into MTT assay for Viability.
2. MTT Assay for Viability: A MTT Working Solution of 1 mg/mL MTT (Methylthiazolyldiphenyl-tetrazolium bromide) is prepared to place in tissue culture plates by diluting two 2 mL vials of the 5 mg/mL MTT concentrate (MatTek) with two 8 mL tubes of MTT Diluent (MatTek) in a 50 mL sterile centrifuge tube and spinning at 1000 RPM in a Brinkman centrifuge. The resulting supernate is decanted into a fresh tube, wrapped in foil, and stored at 4° C. between assays. Prior to the designated harvest time, 24-well tissue culture plates are prepared for the MTT assay by pipetting 300 uL of the MTT Working Solution into each well and allowing to equilibrate to room temperature. The 24 well tissue culture plates containing MTT working solution are protected from light during preparation and the assay. Following the designated exposure time, each of the 6 well tissue culture plates are rinsed with Dulbecco's PBS (DPBS, MatTek) using a sterile squirt bottle and directing the stream at the edges of the 6 well tissue culture plates insert to create a vortex inside the insert to remove residual composition. Each tissue culture is inverted and tapped on paper toweling to remove excess DPBS, then placed into individual wells of the 24-well tissue culture plate containing MTT Working Solution. Tissues cultures exposed for only 15 minutes are placed into the MTT solution following completion of their post-exposure incubation. Following placement into the MTT, the tissue cultures are incubated for three hours in the tissue culture incubator. During the incubation, extraction plates are prepared to extract the MTT from the tissue cultures, by pipetting 2 mL of MTT Extractant (acidified isopropanol) (MatTek) into each well. After incubation, each tissue culture is removed from the MTT solution, blotted onto paper toweling, and into a well of the extractant solution. Following harvest of all the tissue cultures, the extraction plate is wrapped with Parafilm and aluminum foil, placed into a zipper-lock plastic bag, and placed into the refrigerator until read. On the day of reading, each tissue culture insert is removed from the plate and its contents decanted back into the wells of the extraction plate. Extractants are pipetted from each well (200 uL, in duplicate) and transferred into a 96-well clear flat bottom assay plate. The 96-well clear flat bottom assay plates are read at λ=570 nm on the Molecular Devices VMax plate reader, using MTT Extractant as the blank. The % Viability is calculated for each composition from the MTT absorbance readings as follows:

$$\% Viability = \frac{OD 570 composition nm}{Mean OD 570 of water control nm} \times 100$$

3. IL1α ELISA: Assay Medium collected from each tissue culture at the time of harvest and MTT assay for Viability, and is stored frozen at −20° C. until quantified in the IL1α ELISA. An ELISA kit (R&D Systems, Catalog SLA50) is used for the assay, using the manufacturer's protocol. The assay medium is evaluated at a 1:20 dilution, and undiluted. The protocol is provided below. All materials are provided with the assay kit.
   a. Reagent preparation:
      i. Allow all reagents and materials to come to room temperature prior to use.
      ii. Wash buffer: dilute 20 mL of Wash Buffer Concentrate with 480 mL MilliQ water
      iii. IL1α standard: Reconstitute the IL1α standard with 5 mL of Calibrator Diluent RD5. Make serial 1:2 dilutions of the standard in calibrator diluent.
      iv. Substrate solution: Mix 10 mL Color Reagent A and Color Reagent B, within 15 minutes of use.
   b. Add 50 uL of Assay Diluent RD1C to each well of a culture plate. Add 200 uL standard, sample, or sample dilution to each well. Incubate 2 hours at room temperature.
   c. Decant the contents of the plate and wash 3 times with 200 uL/well of Wash Buffer. Blot the plate onto a paper towel.
   d. Add 200 uL Conjugate to each well. Incubate 1 hour at room temperature.
   e. Decant the contents of the plate and wash 3 times with 200 uL/well of Wash Buffer. Blot the plate onto a paper towel.
   f. Add 200 uL substrate solution to each well. Incubate 20 minutes at room temperature. Protect from light.
   g. Add 50 uL stop solution to each well. Read at 450 nm on a VMax plate reader (Molecular Devices) within 30 minutes.
   h. The IL1α concentration from each sample is determined by fitting the standard curve with a 4-parameter logistic curve fit using plate reader software (SoftMax, Molecular Devices), and calculating the unknowns from the generated parameters.
   i. IL1α is normalized to the % Viability result from the MTT assay to account for Viability loss, using the following formula:

$$IL 1 α pg \text{/} ml = \frac{Sample IL 1 α pg \text{/} ml}{Sample \% Viability} \times 100$$

4. IL8 ELISA: Assay medium is collected from each tissue culture at the time of harvest and MTT assay, and is stored frozen at −20° C. until quantified in the IL8 ELISA. An ELISA kit (R&D Systems, Catalog D8000C) is used for the assay, using the manufacturer's protocol. The assay medium is evaluated at a 1:20 dilution, and undiluted. The protocol is provided below. All materials are provided with the assay kit.
   a. Reagent preparation:
      i. Allow all reagents and materials to come to room temperature prior to use.
      ii. Wash buffer: dilute 20 mL of Wash Buffer Concentrate with 480 mL MilliQ water
      iii. IL8 standard: Reconstitute the IL8 standard with 5 mL of Calibrator Diluent RD5. Make serial 1:2 dilutions of the standard in calibrator diluent.
      iv. Substrate solution: Mix 10 mL Color Reagent A and Color Reagent B, within 15 minutes of use.
   b. Add 100 uL of Assay Diluent RD1-85 to each well of a culture plate. Add 50 uL standard, sample, or sample dilution to each well. Incubate 2 hours at room temperature.
   c. Decant the contents of the plate and wash 4 times with 200 uL/well of Wash Buffer. Blot the plate onto a paper towel.
   d. Add 100 uL Conjugate to each well. Incubate 1 hour at room temperature.
   e. Decant the contents of the plate and wash 3 times with 200 uL/well of Wash Buffer. Blot the plate onto a paper towel.
   f. Add 200 uL substrate solution to each well. Incubate 20 minutes at room temperature. Protect from light.
   g. Add 50 uL stop solution to each well. Read at 450 nm on a VMax plate reader (Molecular Devices) within 30 minutes.
   h. The IL8 concentration from each sample is determined by fitting the standard curve with a 4-parameter logistic curve fit using plate reader software (SoftMax, Molecular Devices), and calculating the unknowns from the generated parameters.
   i. IL8 is normalized to the % Viability result from the MTT assay to account for viability loss, using the following formula:

$$IL 8 pg \text{/} ml = \frac{Sample IL 8 pg \text{/} ml}{Sample \% Viability} \times 100$$

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the leave on composition, unless otherwise specified.

| | Example | Example | Example | Example | Example | Example |
|---|---|---|---|---|---|---|
| Raw Material | 1 wt. % | 2 wt. % | 3 wt. % | 4 wt. % | 5 wt. % | 6 wt. % |
| Polyoxyethylene (20) | 0.347 | 0.347 | 0.347 | 0.347 | 0.347 | 0.347 |
| Isohexadecyl Ether | | | | | | |
| Polyacrylic Acid | 0.1 | x | 0.1 | 0.10 | 0.10 | 0.1 |
| Propylene Glycol | 0.15 | 0.15 | 0.15 | 0.15 | 0.15 | 0.15 |
| Fragrance | 0.015 | 0.015 | 0.015 | 0.015 | 0.015 | 0.015 |
| DMDM Hydantoin | 0.2 | 0.2 | 0.2 | 0.2 | 0.2 | 0.2 |
| Sodium Benzoate | 0.2 | 0.2 | 0.2 | 0.2 | 0.2 | 0.2 |
| Poloxamer 184 | 1.0 | 1.0 | 1.0 | 1.0 | 1.0 | x |
| Poloxyethylene 4 Sorbitan | 0.5 | 0.5 | 0.5 | 0.5 | 0.5 | 1.0 |
| Monolaurate | | | | | | |
| Divinyldimethicone/Dimethicone | 0.3 | x | x | x | x | x |
| Copolymer | | | | | | |
| Amodimethicone | x | 0.3 | x | x | x | x |
| Dimethicone | x | x | 0.3 | x | x | x |
| Dimethiconol | x | x | x | x | x | x |
| Silicone Quaternium-16 | x | x | x | 0.3 | x | x |
| Alkylmethyl Siloxane Copolyol | x | x | x | x | 0.3 | x |
| Polydimethylsiloxane | 0.1 | 0.1 | 0.1 | 0.1 | 0.1 | 0.1 |
| Aloe Vera Gel | 0.2 | 0.2 | 0.2 | 0.2 | 0.2 | 0.2 |
| beta Cyclodextrin | 0.1 | 0.1 | 0.1 | 0.1 | 0.1 | 0.1 |
| Triethanolamine | 0.1 | 0.1 | 0.1 | 0.1 | 0.1 | 0.1 |
| Polysorbate 20 | x | x | x | x | x | x |
| Oleth-10 | x | x | x | x | x | x |
| Disodium Laureth Sulfosuccinate | x | x | x | x | x | 0.5 |
| 4-chloro-3,5-dimethylphenol | x | x | x | x | x | x |
| Laureth-23 | x | x | x | x | x | x |
| Ajidew NL-50 | x | x | x | x | x | x |
| Hydroxypropyl beta Cyclodextrin | x | x | x | x | x | x |
| Sodium Alkyl Glyceryl Sulfonate | x | x | x | x | x | x |
| Sodium Methyl-2 Sulfo C12–C18 | x | x | x | x | x | x |
| Ester | | | | | | |
| Salicylic Acid | x | x | x | x | x | x |
| Citric Acid | x | x | x | x | x | x |
| Polyalkyleneoxide | x | x | x | x | x | x |
| Polydimethylsiloxane | | | | | | |
| Potassium Sorbate | x | x | x | x | x | x |
| Poloxamer 333 | x | x | x | x | x | x |
| PEG-6 Caprylic/Capric | x | x | x | x | x | x |
| Glycerides | | | | | | |
| PPG-12 PEG-50 Lanolin | x | x | x | x | x | x |
| Water | qs to | qs to | qs to | qs to | qs to | qs to |
| | 100% | 100% | 100% | 100% | 100% | 100% |
| | Example | Example | Example | Example | Example | Example |
| Raw Material | 7 wt. % | 8 wt. % | 9 wt. % | 10 wt. % | 11 wt. % | 12 wt. % |
| Polyoxyethylene (20) | 0.347 | 0.347 | 0.347 | 0.347 | 0.347 | 0.347 |
| Isohexadecyl Ether | | | | | | |
| Polyacrylic Acid | 0.1 | 0.1 | 0.1 | x | 0.1 | x |
| Propylene Glycol | 0.15 | 0.15 | 0.1 | 0.15 | 0.15 | 0.15 |
| Fragrance | 0.015 | 0.015 | 0.1 | 0.075 | 0.015 | 0.015 |
| DMDM Hydantoin | 0.2 | 0.2 | 0.2 | x | 0.2 | 0.2 |
| Sodium Benzoate | 0.2 | 0.2 | x | 0.25 | 0.2 | 0.2 |
| Poloxamer 184 | 1.0 | 1.0 | x | x | 1.0 | x |
| Poloxyethylene 4 Sorbitan | x | x | x | x | 0.5 | x |
| Monolaurate | | | | | | |
| Divinyldimethicone/Dimethicone | x | 0.3 | x | x | x | 0.5 |
| Copolymer | | | | | | |
| Amodimethicone | x | x | x | x | x | x |
| Dimethicone | 0.3 | x | x | x | x | x |
| Dimethiconol | x | x | x | x | 0.3 | x |
| Silicone Quaternium-16 | x | x | x | x | x | x |
| Alkylmethyl Siloxane Copolyol | x | x | x | x | x | x |
| Polydimethylsiloxane | 0.1 | x | x | x | 0.1 | x |
| Aloe Vera Gel | 0.2 | x | 0.2 | 0.2 | 0.2 | 0.2 |
| beta Cyclodextrin | 0.1 | 0.05 | 0.3 | 0.05 | 0.1 | 0.1 |
| Triethanolamine | 0.1 | 0.2 | 0.2 | 0.2 | 0.1 | 0.1 |
| Polysorbate 20 | x | x | x | x | x | 1.0 |
| Oleth-10 | x | x | x | x | x | 0.5 |
| Disodium Laureth Sulfosuccinate | x | x | x | x | x | x |
| 4-chloro-3,5-dimethylphenol | x | x | 0.1 | 0.1 | x | x |
| Laureth-23 | x | 0.25 | x | 0.25 | x | x |
| Ajidew NL-50 | x | x | 0.2 | 0.4 | x | x |
| Hydroxypropyl beta Cyclodextrin | x | x | 3.0 | 3.0 | x | x |
| Sodium Alkyl Glyceryl Sulfonate | x | x | 0.86 | 0.172 | x | x |
| Sodium Methyl-2 Sulfo C12–C18 | x | x | 0.25 | 0.272 | x | x |
| Ester | | | | | | |
| Salicylic Acid | x | x | x | 0.2 | x | x |
| Citric Acid | x | x | x | 0.1 | x | x |
| Polyalkyleneoxide | x | x | 0.25 | x | x | x |
| Polydimethylsiloxane | | | | | | |
| Potassium Sorbate | x | x | 0.2 | x | x | x |
| Poloxamer 333 | 0.5 | 0.5 | x | x | x | x |
| PEG-6 Caprylic/Capric | x | 0.5 | x | x | x | x |
| Glycerides | | | | | | |
| PPG-12 PEG-50 Lanolin | x | 0.5 | x | x | x | x |
| Water | qs to | qs to | qs to | qs to | qs to | qs to |
| | 100% | 100% | 100% | 100% | 100% | 100% |
| | Example | Example | Example | Example | Example | Example |
| Raw Material | 13 wt. % | 14 wt % | 15 wt. % | 16 wt. % | 17 wt. % | 18 wt % |
| Polyoxyethylene (20) | 0.347 | 0.347 | 0.347 | 0.347 | 0.347 | 0.347 |
| Isohexadecyl Ether | | | | | | |
| Polyacrylic Acid | 0.1 | 0.1 | 0.10 | 0.1 | 0.1 | 0.1 |
| Propylene Glycol | 0.15 | 0.15 | 0.15 | 0.15 | 0.15 | 0.15 |
| Fragrance | 0.015 | 0.015 | 0.015 | 0.015 | 0.015 | 0.015 |
| DMDM Hydantoin | 0.2 | 0.2 | 0.2 | 0.2 | 0.20 | 0.2 |
| Sodium Benzoate | 0.2 | 0.2 | 0.2 | 0.2 | 0.20 | 0.2 |
| Poloxamer 184 | 15.0 | 25.0 | 40.0 | 50.0 | 60.0 | 1.0 |
| Poloxyethylene 4 Sorbitan | 5.0 | 15.0 | 20.0 | 25.0 | 30.0 | 0.5 |
| Monolaurate | | | | | | |
| Divinyldimethicone/Dimethicone | 0.3 | 0.3 | 0.3 | 0.3 | 0.3 | 5.0 |
| Copolymer | | | | | | |
| Amodimethicone | x | x | x | x | x | x |
| Dimethicone | x | x | x | x | x | x |
| Dimethiconol | x | x | x | x | x | x |
| Silicone Quaternium-16 | x | x | x | x | x | x |
| Alkylmethyl Siloxane Copolyol | x | x | x | x | x | x |
| Polydimethylsiloxane | 2.0 | 2.0 | 2.0 | 2.0 | 2.0 | 0.1 |
| Aloe Vera Gel | 0.2 | 0.2 | 0.2 | 0.2 | 0.2 | 0.2 |
| beta Cyclodextrin | 0.1 | 0.1 | 0.1 | 0.1 | 0.1 | 0.1 |
| Triethanolamine | 0.1 | 0.1 | 0.1 | 0.1 | 0.1 | 0.1 |
| Polysorbate 20 | x | x | x | x | x | x |
| Oleth-10 | x | x | x | x | x | x |
| Disodium Laureth Sulfosuccinate | x | x | x | x | x | x |
| 4-chloro-3,5-dimethylphenol | x | x | x | x | x | x |
| Laureth-23 | x | x | x | x | x | x |
| Ajidew NL-50 | x | x | x | x | x | x |
| Hydroxypropyl beta | x | x | x | x | x | x |
| Cyclodextrin | | | | | | |
| Sodium Alkyl Glyceryl | x | x | x | x | x | x |
| Sulfonate | | | | | | |
| Sodium Methyl-2 Sulfo C12–C18 | x | x | x | x | x | x |
| Ester | | | | | | |
| Salicylic Acid | x | x | x | x | x | x |
| Citric Acid | x | x | x | x | x | x |
| Polyalkyleneoxide | x | x | x | x | x | x |
| Polydimethylsiloxane | | | | | | |
| Potassium Sorbate | x | x | x | x | x | x |
| Poloxamer 333 | x | x | x | x | x | x |
| PEG-6 Caprylic/Capric | x | x | x | x | x | x |
| Glycerides | | | | | | |
| PPG-12 PEG-50 Lanolin | x | x | x | x | x | x |
| Water | qs to | qs to | qs to | qs to | qs to | qs to |
| | 100% | 100% | 100% | 100% | 100% | 100% |
| | Example | Example | Example | Example | Example | Example |
| Raw Material | 19 wt. % | 20 wt. % | 21 wt. % | 22 wt % | 23 wt. % | 24 wt. % |
| Polyoxyethylene (20) | 0.347 | 0.347 | 0.347 | 0.347 | 0.347 | 0.347 |
| Isohexadecyl Ether | | | | | | |
| Polyacrylic Acid | 0.1 | 0.1 | 0.1 | 0.1 | 0.1 | 0.1 |
| Propylene Glycol | 0.15 | 0.15 | 0.15 | 0.15 | 0.15 | 0.15 |
| Fragrance | 0.015 | 0.015 | 0.015 | 0.015 | 0.015 | 0.015 |
| DMDM Hydantoin | 0.2 | 0.2 | 0.2 | 0.2 | 0.2 | 0.2 |
| Sodium Benzoate | 0.2 | 0.2 | 0.2 | 0.2 | 0.2 | 0.2 |
| Poloxamer 184 | 1.0 | 1.0 | 1.0 | 1.0 | 1.0 | 1.0 |
| Poloxyethylene 4 Sorbitan | 0.5 | 0.5 | 0.5 | 0.5 | 0.5 | 0.5 |
| Monolaurate | | | | | | |
| Divinyldimethicone/Dimethicone | 20.0 | 50.0 | 75.0 | 0.3 | 0.3 | 0.3 |
| Copolymer | | | | | | |
| Amodimethicone | x | x | x | x | x | x |
| Dimethicone | x | x | x | x | x | X |
| Dimethiconol | x | x | x | x | x | X |
| Silicone Quaternium-16 | x | x | x | x | x | X |
| Alkylmethyl Siloxane Copolyol | x | x | x | x | x | X |
| Polydimethylsiloxane | 0.1 | 0.1 | 0.1 | 0.1 | 0.1 | 0.1 |
| Aloe Vera Gel | 0.2 | 0.2 | 0.2 | 5.0 | 20.0 | 50.0 |
| beta Cyclodextrin | 0.1 | 0.1 | 0.1 | 0.1 | 0.1 | 0.1 |
| Triethanolamine | 0.1 | 0.1 | 0.1 | 0.1 | 0.1 | 0.1 |
| Polysorbate 20 | x | x | x | x | x | x |
| Oleth-10 | X | x | x | x | x | X |
| Disodium Laureth Sulfosuccinate | X | x | x | x | x | X |
| 4-chloro-3,5-dimethylphenol | X | x | x | x | x | X |
| Laureth-23 | X | x | x | x | x | X |
| Ajidew NL-50 | X | x | x | x | x | x |
| Hydroxypropyl beta | X | x | x | x | x | x |
| Cyclodextrin | | | | | | |
| Sodium Alkyl Glyceryl | X | x | x | x | x | x |
| Sulfonate | | | | | | |
| Sodium Methyl-2 Sulfo C12–C18 | X | x | x | x | x | x |
| Ester | | | | | | |
| Salicylic Acid | X | x | x | x | x | x |
| Citric Acid | X | x | x | x | x | x |
| Polyalkyleneoxide | X | x | x | x | x | x |
| Polydimethylsiloxane | | | | | | |
| Potassium Sorbate | X | x | x | x | x | x |
| Poloxamer 333 | X | x | x | x | x | x |
| PEG-6 Caprylic/Capric | X | x | x | x | x | x |
| Glycerides | | | | | | |
| PPG-12 PEG-50 Lanolin | X | x | x | x | x | x |
| Water | qs to | qs to | qs to | qs to | qs to | qs to |
| | 100% | 100% | 100% | 100% | 100% | 100% |

| | Example |
|---|---|
| Raw Material | 25 wt. % |
| Polyoxyethylene (20) Isohexadecyl | 0.347 |
| Ether | |
| Polyacrylic Acid | 0.1 |
| Propylene Glycol | 0.15 |
| Fragrance | 0.015 |
| DMDM Hydantoin | 0.2 |
| Sodium Benzoate | 0.2 |
| Poloxamer 184 | 1.0 |
| Poloxyethylene 4 Sorbitan | 0.5 |
| Monolaurate | |
| Divinyldimethicone/Dimethicone | 0.3 |
| Copolymer | |
| Amodimethicone | X |
| Dimethicone | X |
| Dimethiconol | X |
| Silicone Quaternium-16 | X |
| Alkylmethyl Siloxane Copolyol | X |
| Polydimethylsiloxane | 0.1 |
| Aloe Vera Gel | 75.0 |
| beta Cyclodextrin | 0.1 |
| Triethanolamine | 0.1 |
| Polysorbate 20 | X |
| Oleth-10 | X |
| Disodium Laureth Sulfosuccinate | X |
| 4-chloro-3,5-dimethylphenol | X |
| Laureth-23 | X |
| Ajidew NL-50 | X |
| Hydroxypropyl beta Cyclodextrin | X |
| Sodium Alkyl Glyceryl Sulfonate | X |
| Sodium Methyl-2 Sulfo C12–C18 | X |
| Ester | |
| Salicylic Acid | X |
| Citric Acid | X |
| Polyalkyleneoxide | X |
| Polydimethylsiloxane | |
| Potassium Sorbate | X |
| Poloxamer 333 | X |
| PEG-6 Caprylic/Capric Glycerides | X |
| PPG-12 PEG-50 Lanolin | X |
| Water | qs to |
| | 100% |

### Examples 1-25

Examples 1-25 may be prepared using conventional formulation and mixing techniques.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed:

1. A leave on companion animal composition comprising; a composition having a Total Lather Volume of from about 300 ml to about 700 ml.

2. The leave on companion animal composition of claim 1, wherein said composition having a Total Lather Volume of from about 310 ml to about 600 ml.

3. The leave on companion animal composition of claim 1, wherein said composition having a Total Lather Volume of from about 315 ml to about 500 ml.

4. The leave on companion animal composition of claim 1, wherein said composition having a Total Lather Volume of from about 320 ml to about 400 ml.

5. The leave on companion animal composition of claim 1, wherein said composition having a Total Lather Volume of from about 325 ml to about 380 ml.

6. The leave on companion animal composition of claim 1, further comprising a surfactant component.

7. The leave on companion animal composition of claim 6, comprising from about 0.5% to about 95%, by weight of the composition, of said surfactant component.

8. The leave on companion animal composition of claim 6, wherein said surfactant component is selected from the group consisting of anionic surfactant, non-ionic surfactant, amphoteric surfactant, cationic surfactant, soap, and mixtures thereof.

9. The leave on companion animal composition of claim 6, wherein said anionic surfactant is selected from the group consisting of sarcosinates, sulfates, ethoxylated sulfate, sulfonates, glyceryl sulfonates, isethionates, phosphates, taurates, lactylates, glutamates, soaps, sulfosuccinates, ethoxylated sulfosuccinates, and mixtures thereof.

10. The leave on companion animal composition of claim 6, wherein said nonionic surfactant is selected from the group consisting of alkyl glucosides, polyglucosides, polyhydroxy fatty acid amides, alkoxylated fatty acid esters, sugar esters,, ethoxylated esters, glycerol esters, ethoxylates, propoxylates, PEG/PPG copolymers, glycerides, sorbitans, and mixtures.

11. The leave on companion animal composition of claim 6, wherein said amphoteric surfactant is selected from the group consisting of amine oxides, betaines, sultaines, hydroxysultaines, alkyliminoacetates, iminodialkanoates, aminoalkanoates, and mixtures thereof.

12. The leave on companion animal composition of claim 1, further comprising conditioning agent selected from the group consisting of silicone polymers, functional silicone polymers, fatty acids, esters of fatty acids, fatty alcohols, ethoxylates, polyol polyesters, glycerine, glycerin mono-esters, glycerin polyesters, epidermal and sebaceous hydrocarbons, lanolin, straight and branched hydrocarbons, vegetable oil, vegetable oil adduct, hydrogenated vegetable oils, nonionic polymers, natural waxes, petrolatum, petrolatum derivatives, synthetic waxes, polyolefinic glycols, polyolefinic monoester, polyolefinic polyesters, cholesterols, cholesterol esters, triglycerides, and mixtures thereof.

13. The leave on companion animal composition of claim 1, further comprising a treatment agent selected from the group consisting of vitamins, cyclodextrins, zeolites, peptides, terpene alcohols, desquamation actives, anti-atrophy actives, anti-oxidants, flavanoids, anti-inflammatory agents, topical anesthetics, chelators, antimicrobial actives, anti-fungal actives, soothing actives, healing actives, moisturizing actives, flea actives, tick actives, other insect actives, and mixtures thereof.

14. The leave on companion animal composition of claim 1, further comprising a foam agent.

15. A leave on companion animal composition comprising; a composition having an IL1α of from about 0 pg/ml to about 7000 pg/ml.

16. The leave on companion animal composition of claim 15, wherein said composition having an IL1α of from about 70 pg/ml to about 6000 pg/ml.

17. The leave on companion animal composition of claim 15, wherein said composition having an IL1α of from about 90 pg/ml to about 5000 pg/ml.

18. The leave on companion animal composition of claim 15, wherein said composition having an IL1α of from about 100 pg/ml to about 4000 pg/ml.

19. The leave on companion animal composition of claim 15, further comprising a surfactant component.

20. The leave on companion animal composition of claim 19, comprising from about 0.5% to about 95%, by weight of the composition, of said surfactant component.

21. The leave on companion animal composition of claim 19, wherein said surfactant component is selected from the group consisting of anionic surfactant, non-ionic surfactant, amphoteric surfactant, cationic surfactant, soap, and mixtures thereof.

22. The leave on companion animal composition of claim 21, wherein said anionic surfactant is selected from the group consisting of sarcosinates, sulfates, ethoxylated sulfate, sulfonates, glyceryl sulfonates, isethionates, phosphates, taurates, lactylates, glutamates, soaps, sulfosuccinates, ethoxylated sulfosuccinates, and mixtures thereof.

23. The leave on companion animal composition of claim 21, wherein said nonionic surfactant is selected from the group consisting of alkyl glucosides, polyglucosides, polyhydroxy fatty acid amides, alkoxylated fatty acid esters, sugar esters, ethoxylated esters, glycerol esters, ethoxylates, propoxylates, PEG/PPG copolymers, glycerides, sorbitans, and mixtures.

24. The leave on companion animal composition of claim 21, wherein said amphoteric surfactant is selected from the group consisting of amine oxides, betaines, sultaines, hydroxysultaines, alkyliminoacetates, iminodialkanoates, aminoalkanoates, and mixtures thereof.

25. The leave on companion animal composition of claim 15, further comprising conditioning agent selected from the group consisting of silicone polymers, functional silicone polymers, fatty acids, esters of fatty acids, fatty alcohols, ethoxylates, polyol polyesters, glycerine, glycerin mono-esters, glycerin polyesters, epidermal and sebaceous hydrocarbons, lanolin, straight and branched hydrocarbons, vegetable oil, vegetable oil adduct, hydrogenated vegetable oils, nonionic polymers, natural waxes, petrolatum, petrolatum derivatives, synthetic waxes, polyolefinic glycols, polyolefinic monoester, polyolefinic polyesters, cholesterols, cholesterol esters, triglycerides, and mixtures thereof.

26. The leave on companion animal composition of claim 15, further comprising a treatment agent selected from the group consisting of vitamins, cyclodextrins, zeolites, peptides, terpene alcohols, desquamation actives, anti-atrophy actives, anti-oxidants, flavanoids, anti-inflammatory agents, topical anesthetics, chelators, antimicrobial actives, anti-fungal actives, soothing actives, healing actives, moisturizing actives, flea actives, tick actives, other insect actives, and mixtures thereof.

27. The leave on companion animal composition of claim 15, further comprising a foam agent.

28. A leave on companion animal composition comprising; a composition having a Total Lather Volume of from about 300 ml to about 700 ml;
said composition having IL1α of from about 0 pg/ml to about 7000 pg/ml; and
said composition having a Friction Coefficient of from about 1.2 to about 2.

29. An article of commerce comprising: a container comprising; a leave on companion animal composition, which provides conditioning, cleansing and treatment benefits to a companion animal when applied and comprises:
(a) a composition having a Total Lather Volume from about 300 ml to about 700 ml;
(b) said composition having IL1α of from about 0 pg/ml to about 7000 pg/ml;
wherein said container has instructions for conditioning, cleansing, and treating the companion animal, comprising the instructions to wash as normal, massage said companion animal, and optionally pat dry with a towel.
